(19) United States
(12) Patent Application Publication
(10) Pub. No.: US 2018/0193401 A1
(43) Pub. Date: Jul. 12, 2018

(54) ANTI-OBESITY COMPOSITION COMPRISING NATURAL COMPLEX

(71) Applicants: Sung Yeoun HWANG, Gyeongsangbuk-do (KR); MEGABIOWOOD CO., LTD., Jeollanam-do (KR)

(72) Inventor: Sung Yeoun HWANG, Gyeongsangbuk-do (KR)

(73) Assignees: MEGABIOWOOD CO., LTD.; Sung Yeoun Hwang

(21) Appl. No.: 15/869,231

(22) Filed: Jan. 12, 2018

(30) Foreign Application Priority Data
Jan. 12, 2017 (KR) 10-2017-0004974

(51) Int. Cl. A61K 36/899; A61P 3/04; A61K 36/73

(52) U.S. Cl. CPC A61K 36/899; A61K 2236/33; A61K 36/73; A61P 3/04

(57) ABSTRACT

A pharmaceutical composition and a health food composition for preventing, treating and improving obesity, which include an extract complex of bamboo leaves and quince as an active ingredient, are disclosed. Because the extract complex has an excellent anti-obesity effect without causing any side effects, the extract complex can be effectively used in a pharmaceutical composition and health food for preventing, improving and treating obesity. Therefore, a novel therapeutic agent whose effect goes beyond conventional natural anti-obesity drugs and a novel therapy can be provided.

### CROSS-REFERENCE TO RELATED APPLICATION

This application claims priority to and the benefit of Korean Patent Application No. 2017-0004974, filed on Jan. 12, 2017, the disclosure of which is incorporated herein by reference in its entirety.

### FIELD

The present disclosure relates to a pharmaceutical composition and a health food composition for preventing, treating and improving obesity, which includes an extract complex of bamboo leaves and quince as an active ingredient.

### BACKGROUND

Obesity is one of the most common nutrition disorders around the world, and refers to a condition in which extra calories accumulate in the form of fat in the body through the intake of excessive calories, compared to calories to be consumed. That is, obesity is a metabolic disease that is caused due to the imbalance between the intake and consumption of calories, and refers to a condition in which an abnormal increase in adipose tissue is caused due to the intake of excessive calories.

Obesity is considered to be caused by various factors such as hereditary effects, environmental influences caused by the westernized dietary life, psychological effects caused by stress, and the like, but the accurate cause or mechanism of obesity is not clearly established so far.

After the onset of obesity, prolonged obesity may induce various diseases such as hypertension, increased blood cholesterol levels, diabetes, kidney disease, cerebral apoplexy, arteriosclerosis, fatty liver, arthritis, cancer, sleep apnea syndrome, and the like.

Also, it has been known that the obesity is caused by intracellular accumulation of triglycerides (TGs) in adipocytes through differentiation of pre-adipocytes and adipogenesis, and the regulation of such an adipogenesis mechanism is an effective therapy to suppress obesity.

Methods of treating obesity have been proposed, but there is as yet no satisfactory method. Also, such methods have various side effects such as a yo-yo effect in which a person gains back the weight when he/she stops the therapy; an unbalanced nutrition condition caused due to dietary restrictions; infections due to lowered immunity, and the like. Particularly, it has been widely reported that the drug therapies cause side effects such as depression, insomnia, indigestion, and the like. Therefore, there is research being conducted to develop an effective and safe method of promoting weight loss in addition to the conventional methods.

Meanwhile, an anti-obesity composition using a quince extract or a complex extract of bamboo leaves and Scutellaria root is disclosed in the prior art, but an anti-obesity composition using a complex of a quince extract and a bamboo leaf extract is not known.

Quince contains carbohydrates (approximately 13.4 to 20.7%), and a large amount of glucose so that sugar accounts for 10 to 13% of the carbohydrates. Therefore, an increase in glucose concentration of a single quince extract results in an increase in viscosity of the extract. In this way, the increased viscosity of the extract may cause problems such as a difficulty in preparing quince into formulations and preparations, and the inconvenience of ingesting a large amount of a formulated substance to take an effective dose of the substance because an excessive amount of an excipient should be added to solve this problem.

Also, bamboo leaves have a drawback in that they taste very bitter, which makes it difficult to prepare them into products such as foods, and the like.

### SUMMARY

Accordingly, the present inventors have ardently conducted research to develop natural drugs that are safe and also have an excellent anti-obesity effect without causing any side effects, and found that an extract complex of bamboo leaves and quince has an excellent anti-obesity effect and may also be used to solve the problems of a single extract. Therefore, the present disclosure has been completed based on these facts.

Therefore, the present disclosure is directed to a pharmaceutical composition for preventing or treating obesity, which includes an extract complex of bamboo leaves and quince as an active ingredient.

Also, the present disclosure is directed to a health food composition for preventing or improving obesity, which includes an extract complex of bamboo leaves and quince as an active ingredient.

However, technical problems to be solved by the present disclosure are not limited to the technical problems as described above, and other technical problems not disclosed herein will be clearly understood from the following description by those skilled in the art.

According to an aspect of the present disclosure, there is provided a pharmaceutical composition for preventing or treating obesity, which includes an extract complex of bamboo leaves and quince as an active ingredient.

According to another aspect of the present disclosure, there is provided a health food composition for preventing or improving obesity, which includes an extract complex of bamboo leaves and quince as an active ingredient.

According to still another aspect of the present disclosure, there is provided a method of preventing or treating obesity, which includes administering an extract complex of bamboo leaves and quince into a subject.

According to yet another aspect of the present disclosure, there is provided an anti-obesity use of the extract complex of bamboo leaves and quince.

According to one exemplary embodiment of the present disclosure, the extraction may be performed using water, a C1 to C4 lower alcohol, or a mixed solvent thereof.

According to another exemplary embodiment of the present disclosure, the lower alcohol may be ethanol.

According to still another exemplary embodiment of the present disclosure, the extract complex may be obtained by mixing a bamboo leaf extract and a quince extract at a weight ratio of 1:0.1 to 10.

According to yet another exemplary embodiment of the present disclosure, the weight ratio may be 1:1.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 shows results of evaluating the cytotoxicity of a 30% ethanol extract complex of bamboo leaves plus quince and a hot-water extract complex of bamboo leaves plus quince in 3T3-L1 adipocytes using an MTT assay;

FIG. 2 shows results of measuring a concentration of leptin in 3T3-L1 adipocytes after the 3T3-L1 adipocytes are treated with a varying concentration of each of the 30% ethanol extract complex of bamboo leaves plus quince and the hot-water extract complex of bamboo leaves plus quince;

FIG. 3 shows results of measuring an amount of glycerol production in 3T3-L1 adipocytes after the 3T3-L1 adipocytes are treated with a varying concentration of each of the 30% ethanol extract complex of bamboo leaves plus quince and the hot-water extract complex of bamboo leaves plus quince;

FIG. 4 shows results of measuring a concentration of triglycerides in 3T3-L1 adipocytes after the 3T3-L1 adipocytes are treated with a varying concentration of each of the 30% ethanol extract complex of bamboo leaves plus quince and the hot-water extract complex of bamboo leaves plus quince;

FIGS. 5 and 6 show results of observing a change in body weight after the hot-water extract complex of bamboo leaves plus quince is orally administered to normal diet (ND) and high fat diet (HD) groups of C57BL/6J mice;

FIG. 7 shows results of observing a change in weight of an epididymal adipose tissue (EAT) after the hot-water extract complex of bamboo leaves plus quince is orally administered to the ND and HD groups of C57BL/6J mice;

FIG. 8 shows results of observing a change in weight of a brown adipose tissue (BAT) after the hot-water extract complex of bamboo leaves plus quince is orally administered to the ND and HD groups of C57BL/6J mice;

FIG. 9 shows results of staining a visceral adipose tissue with hematoxylin & eosin (H&E) to observe sizes of the visceral adipose tissue after the hot-water extract complex of bamboo leaves plus quince is orally administered to the ND and HD groups of C57BL/6J mice;

FIG. 10a shows results of measuring the sizes of the visceral adipose tissue after the hot-water extract complex of bamboo leaves plus quince is orally administered to the ND and HD groups of C57BL/6J mice;

FIG. 10b shows results of measuring an area of the visceral adipose tissue after the hot-water extract complex of bamboo leaves plus quince is orally administered to the ND and HD groups of C57BL/6J mice;

FIG. 11 shows results of staining the BAT with hematoxylin & eosin (H&E) to observe sizes of the BAT after the hot-water extract complex of bamboo leaves plus quince is orally administered to the ND and HD groups of C57BL/6J mice;

FIG. 12a shows results of measuring the sizes of the BAT after the hot-water extract complex of bamboo leaves plus quince is orally administered to the ND and HD groups of C57BL/6J mice;

FIG. 12b shows results of measuring an area of the BAT after the hot-water extract complex of bamboo leaves plus quince is orally administered to the ND and HD groups of C57BL/6J mice;

FIG. 13 shows results of staining the EAT with hematoxylin & eosin (H&E) to observe sizes of the EAT after the hot-water extract complex of bamboo leaves plus quince is orally administered to the ND and HD groups of C57BL/6J mice;

FIG. 14a shows results of measuring the sizes of the EAT after the hot-water extract complex of bamboo leaves plus quince is orally administered to the ND and HD groups of C57BL/6J mice; and

FIG. 14b shows results of measuring an area of the EAT after the hot-water extract complex of bamboo leaves plus quince is orally administered to the ND and HD groups of C57BL/6J mice.

### DETAILED DESCRIPTION

Exemplary embodiments of the present disclosure will be described in detail below with reference to the accompanying drawings. While the present disclosure is shown and described in connection with exemplary embodiments thereof, it will be apparent to those skilled in the art that various modifications can be made without departing from the scope of the invention.

Unless specifically stated otherwise, all the technical and scientific terms used in this specification have the same meanings as what are generally understood by a person skilled in the related art to which the present disclosure belongs. In general, the nomenclature used in this specification and the experimental methods described below is widely known and generally used in the related art.

An extract complex of bamboo leaves and quince according to the present disclosure may be prepared as follows.

An extract complex of the present disclosure may be obtained by washing bamboo leaves and quince with water, drying the bamboo leaves and quince in the shade, grinding each of the bamboo leaves and quince, extracting each of the herbal materials in a volume of water, a C1 to C4 lower alcohol or a mixed solvent thereof, preferably 30% ethanol, which is approximately 1 to 20 times, preferably approximately 2 to 10 times higher than the weight of the herbal materials, at a temperature of 0 to 120° C. for approximately 1 hour to 1 day using an extraction method such as hot-water extraction, cold maceration extraction, reflux condensation extraction, ultrasonic extraction, or supercritical extraction, cooling and filtering the extract, and freeze-drying the filtrate.

The extract complex is preferably obtained by mixing a bamboo leaf extract and a quince extract at a weight ratio of 1:0.1 to 10, and most preferably obtained by mixing the respective single extracts at a weight ratio of 1:1.

A pharmaceutical composition of the present disclosure may further include components such as conventional therapeutically active ingredients, other adjuvants, a pharmaceutically acceptable carrier, and the like. The pharmaceutically acceptable carrier includes saline, sterile water, Ringer's solution, buffered saline, a dextrose solution, a maltodextrin solution, glycerol, and ethanol.

In the present disclosure, the term “subject” refers to a target in need of treatment for a disease, and, more particularly, to a mammal including a human or a non-human primate, a mouse, a rat, a dog, a cat, a horse, cattle, etc. Also, in the present disclosure, the term “pharmaceutically effective amount” may be determined according to factors including the type and severity of a disease to be treated, the age and sex of a patient, the sensitivity to a drug, an administration time, a route of administration, an excretion rate, treatment duration, drugs to be used together, and other factors widely known in the pharmaceutical field. In this case, the pharmaceutically effective amount is an amount that may be determined in consideration of all the factors to obtain the maximum effect without any side effects and thus may be easily determined by those skilled in the art.

The ‘administration method’ may be used without limitation as long as the composition of the present disclosure can reach a target tissue. For example, the administration method encompasses oral administration, intraarterial injection, intravenous injection, percutaneous injection, intranasal administration, transbronchial administration, or intramuscular administration. The composition may be administered daily at a dose of approximately 0.0001 to 100 mg/kg, preferably 0.001 to 10 mg/kg, and may be administered once a day or multiple times in divided doses.

The composition of the present disclosure may be widely used in drugs, foods, and drinks to prevent and improve obesity and may be used in the form of a powder, granules, a pill, a capsule, or a beverage.

In the present disclosure, the term “bamboo leaves” refers to leaves of Phyllostachys bambusoides Siebold & Zucc., Phyllostachys pubescens Mazel ex Lehaie., Phyllostachys nigra var. henonis Stapf, Sasa borealis (Hack.) Makino & Shibata, and the like, all of which belong to the family Bambusaceae, and is known to reduce a fever, eliminate dysphoria, generate a body fluid, and have a diuretic effect in oriental medicine.

The “quince” is native to China, and a deciduous shrub or small tree that belongs to the genus Chaenomelis Lindl. of the family Rosaceae. In oriental medicine, the quince has been prescribed as a medicine effective against acute stomach problems, beriberi, myalgia, arthritis, neuralgia, and the like because quince has been recognized as a drug for harmonizing the stomach and removing moisture. Also, the quince has been known to be effective against coughs, phlegm, pneumonia, bronchitis, and the like.

In the present disclosure, the term “obesity” generally refers to a condition in which an excessive amount of an adipose tissue is present in the body or a body obesity index (i.e., a body mass index: a value obtained by dividing a body weight (kg) by the square of a height (m)) is greater than or equal to 25. In a subject who develops the symptoms of obesity, fatty acids and glucose flowing from plasma into adipocytes are generally esterified and accumulated in the form of triglycerides.

In the present disclosure, an effect of an extract complex of bamboo leaves plus quince was checked to search for an anti-obesity effect by differentiating 3T3-L1 adipocytes as an obese cell model and treating the 3T3-L1 adipocytes with the extract complex of bamboo leaves plus quince to determine cell viability, triglyceride content, leptin production, glycerol release, and the like. Also, after the extract complex of bamboo leaves plus quince was orally administered to C57BL/6J mice of an animal model for 8 weeks while a normal diet (ND) and a high fat diet (HD) are being provided to the C57BL/6J mice, the anti-obesity effect was verified through changes in body weights, blood lipids and adipose tissues of the C57BL/6J mice.

As a result, it was confirmed that the extract complex of bamboo leaves plus quince serves to reduce the accumulation of lipids and increase glycerol secretion in the 3T3-L1 adipocytes, and suppress the differentiation and synthesis of adipocytes. Also, a significant body weight loss effect was exhibited in the C57BL/6J mice to which the extract complex of bamboo leaves plus quince was orally administered for 8 weeks. It was revealed that such a body weight loss effect is due to a decrease in weight of the adipose tissues, and a decrease in size and area of the adipocytes when the extract complex of bamboo leaves plus quince is administered. Also, it was seen that blood lipid concentration is significantly improved by a decrease in body fat.

Therefore, the extract complex of bamboo leaves plus quince according to the present disclosure may be effectively used to prevent and treat obesity, and an effect of the extract complex is not significantly different from that of a garcinia cambogia extract (HCA) which is currently used as a health functional food for reducing body fat. Accordingly, the extract complex of bamboo leaves plus quince may be proposed as a novel candidate for the health functional food for reducing body fat.

Hereinafter, preferred examples are provided to aid in understanding the present disclosure. However, it should be understood that detailed description provided herein is merely intended to provide a better understanding of the present disclosure and is not intended to limit the scope of the present disclosure.

### EXAMPLES

### Example 1: Experimental Method

1-1. Preparation of Extract Complex

Selected materials purchased from Dong Yang Herb Co., Ltd. were used as the natural materials used in this experiment. First, each of bamboo leaves and quince was dried in the shade, and ground. Thereafter, each of 50 g of the bamboo leaves and 50 g of the quince was added to 1 L of 30% ethanol, and extracted in a boiling pot to obtain a hot-water extract, which was then freeze-dried. 100 mg of the freeze-dried sample was added to and dissolved in 1 mL of 30% ethanol, and then filtered through a 0.2 μm filter to remove impurities. Then, the resulting filtrate was used as the sample.

A dose of the extract complex (obtained by mixing a bamboo leaf extract and a quince extract at a weight ratio of 1:1) which an adult weighing 60 kg took was set as 1,000 mg or 3,000 mg, and feed was prepared using the following equation in order to administer the extract complex to mice.

HED (mg/kg)=Animal dose(mg/kg)×Animal Km/Human Km

1-2. Cell Culture

An adipocyte 3T3-L1 cell line was purchased from the American Type Culture Collection (Rockville, Md., USA), and cultured at 37° C. in a DMEM (Invitrogen, USA) medium supplemented with 10% fetal bovine serum (FBS; Invitrogen, USA) in a 5% CO₂ incubator. To avoid contamination, 100 units/mL of penicillin and 100 μg/mL of streptomycin (Gibco/BRL, USA) as antibiotics were added thereto, and the cells were treated with trypsin-EDTA (Gibco/BRL, USA), and sub-cultured. The medium was replaced with a fresh one every two or three days.

For treatment with the extract complex, the cultured 3T3-L1 cell line was transferred to a 24-well plate (Corning, USA) at a density of 1×10⁵ cells/well. After the medium was replaced with a FBS-free medium the next evening, the 3T3-L1 cell line was treated for 16 hours.

1-3. Laboratory Animals

Four-week-old male C57BL/6J mice were purchased from Central Lab-Animal Inc. (Seoul, Korea), adapted for a week to a normal diet in which fats accounted for 10% of the total calories, and then used for experiments.

1-4. Mouse Animal Model Experimental Groups

To induce obesity, a high fat diet in which fats accounted for 60% of the total calories was supplied to all groups other than a normal diet (ND) group and an ND sample group, and a normal diet in which fats accounted for 10% of the total calories was freely supplied to the ND group and the ND sample group for a total of 8 weeks.

The experimental groups were divided into a total of 6 groups: a ND group, a high fat diet (HD) group, a positive control (HCA), a sample group (ND+JMW) in which a hot-water extract complex of bamboo leaves plus quince was administered to the ND group, and sample-efficacy groups (HD+JMW200 and HD+JMW400) in which a two-fold concentration of the hot-water extract complex of bamboo leaves plus quince was administered to the high fat diet group. In this case, ten mice were assigned to each group using a randomized block design, and used for experiments.

To check an anti-obesity effect of the sample extract for 8 weeks (week 1 to 8) after a one-week adaptation, the test material was dissolved in distilled water, and orally administered to the positive control, the ND sample group, and the HD sample-efficacy groups once a day at a concentration of 300 mg/kg BW/day, 400 mg/kg BW/day, 200 mg/kg BW/day, and 400 mg/kg BW/day, respectively. Also, distilled water was administered to the ND group and the HD group. In this case, diet compositions for ND and HD are listed in the following Table 1.

TABLE 1

| g/100 g diet | ND | ND + JMW400 | HD | HD + JMW200 | HD + JMW400 | HD + HCA |
|---|---|---|---|---|---|---|
| Casein 80-mesh | 18.96 | 18.96 | 25.84 | 25.84 | 25.84 | 25.84 |
| L-Cysteine | 0.28 | 0.28 | 0.39 | 0.39 | 0.39 | 0.39 |
| Constarch | 29.86 | 29.86 | 0 | 0 | 0 | 0 |
| Maltodextrin 10 | 3.32 | 3.32 | 16.15 | 16.15 | 16.15 | 16.15 |
| Sucrose | 33.17 | 33.17 | 8.89 | 8.89 | 8.89 | 8.89 |
| Cellulose BW200 | 4.74 | 4.74 | 6.46 | 6.46 | 6.46 | 6.46 |
| Soybean oil | 2.37 | 2.37 | 3.23 | 3.23 | 3.23 | 3.23 |
| Lard | 1.90 | 1.90 | 31.66 | 31.66 | 31.66 | 31.66 |
| Mineral mix S10026 | 0.95 | 0.95 | 1.29 | 1.29 | 1.29 | 1.29 |
| Dicalcium phosphate | 1.23 | 1.23 | 1.68 | 1.68 | 1.68 | 1.68 |
| Calcium carbonate | 0.52 | 0.52 | 0.71 | 0.71 | 0.71 | 0.71 |
| Potassium carbonate | 1.56 | 1.56 | 2.13 | 2.13 | 2.13 | 2.13 |
| Vitamin mix V10001 | 0.95 | 0.95 | 1.29 | 1.29 | 1.29 | 1.29 |
| Choline bitartrate | 0.19 | 0.19 | 0.26 | 0.26 | 0.26 | 0.26 |
| Calorie from fat (%) | 10 | 10 | 60 | 60 | 60 | 60 |

ND: normal diet,
HD: high-fat diet,
ND + JMW400: normal diet plus JMW 400 mg/kg BW/day,
HD + JMW200: high-fat diet plus JMW 200 mg/kg BW/day,
HD + JMW400: high-fat diet plus JMW 400 mg/kg BW/day,
HD + HCA: high-fat diet plus HCA 300 mg/kg BW/day.
*JMW was orally administered to mice at a dose of 200 mg/kg BW/day (HD + JMW200), 400 mg/kg BW/day (ND + JMW400, HD + JMW400) and HCA was orally administered to mice at a dose of 300 mg/kg BW/day (HD + HCA300) for 8 weeks.

1-5. Statistics

All measured values were expressed as mean±standard deviation (S.D.), statistical analyses between the respective experimental groups were assayed by one-way ANOVA using the window SPSS program. In this case, a p value was considered to be statistically significant when the p value was less than or equal to 0.05.

### Example 2: In Vitro Cell Experiment

2-1. MTT Assay (Toxicity Test)

The cytotoxicity of the 30% ethanol extract complex of bamboo leaves plus quince and the hot-water extract complex of bamboo leaves plus quince in 3T3-L1 adipocytes was evaluated using a known MTT assay.

As a result, when it was assumed that the cell viability was 100% when only the cells were cultured, the cell viabilities were measured to be 99.84%, 99.15%, 98.44%, 98.57%, 94.50%, and 88.10% when the 30% ethanol extract complex of bamboo leaves plus quince was used at concentrations of 50, 100, 200, 400, 800, and 1,000 μg/mL, respectively, as shown in FIG. 1. Therefore, it was assessed that the 30% ethanol extract complex of bamboo leaves plus quince had no significance when used at a concentration of 50 to 400 μg/mL, and exhibited no cytotoxicity. Also, the cell viabilities were measured to be 99.70%, 98.54%, 99.34%, 98.48%, 95.74%, and 91.55% when the hot-water extract complex of bamboo leaves plus quince was used at concentrations of 50, 100, 200, 400, 800, and 1,000 μg/mL, respectively. That is, it was assessed that the hot-water extract complex of bamboo leaves plus quince had no significance when used at a concentration of 50 to 400 μg/mL, and exhibited no cytotoxicity.

In FIG. 1, Con represents the adipocytes, JME represents the 30% ethanol extract of quince and bamboo leaves, and JMW represents the hot-water extract of quince and bamboo leaves.

Based on these cytotoxicity results, in subsequent experiments, the anti-obesity effect of the extract complex of bamboo leaves plus quince was examined in a concentration range (0 to 400 μg/mL) which did not exhibit cytotoxicity.

2-2. Analysis of Leptin Production

Leptin is a hormone that is produced by an obesity gene in adipocytes. Thus, a blood leptin concentration has been widely used for obesity research as an indicator of body fat mass because a secretion rate of leptin increases with an increase in fat accumulated in the adipocytes. An amount of leptin production in undifferentiated pre-adipocytes was 0.027±0.01 ng/mL, an amount of leptin production in differentiated adipocytes was 0.50±0.01 ng/mL, and an amount of leptin production in the positive control was 0.39±0.01 ng/mL when hydroxycitric acid (HCA) was present at a concentration of 100 μg/mL.

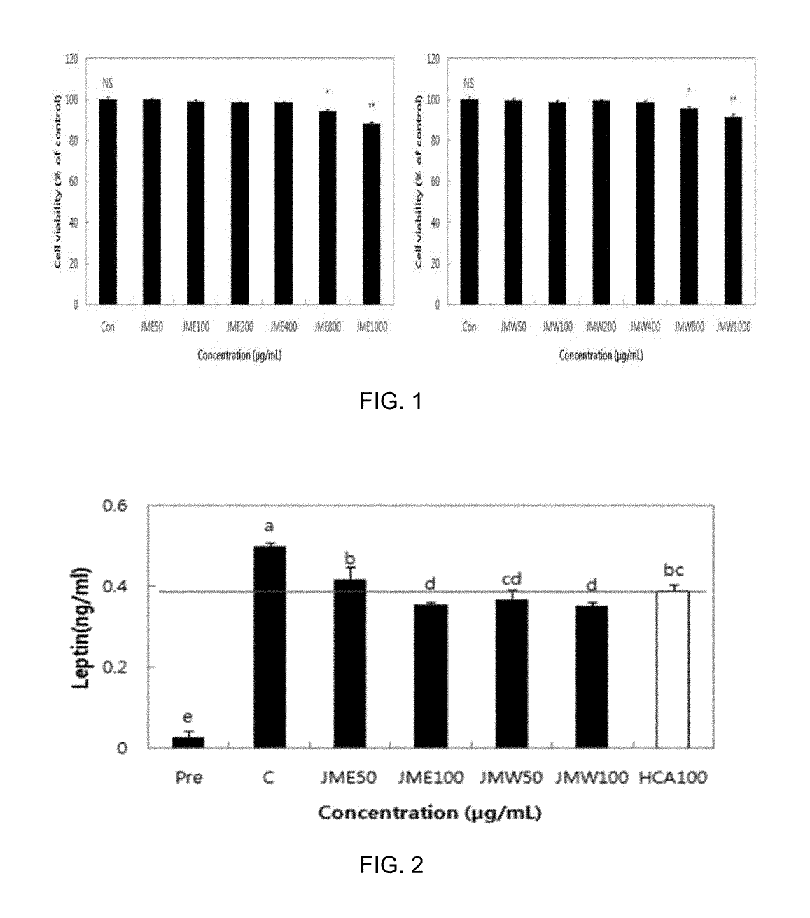

3T3-L1 adipocytes were treated with each of the 30% ethanol extract complex of bamboo leaves plus quince and the hot-water extract complex of bamboo leaves plus quince at concentrations of 50 and 100 μg/mL, and a concentration of leptin was then measured using an ELISA kit. As a result, the amounts of leptin production in the adipocytes treated with the 30% ethanol extract complex of bamboo leaves plus quince and the hot-water extract complex of bamboo leaves plus quince decreased significantly (p<0.05) to 0.42±0.03 and 0.35±0.01 ng/mL, and 0.37±0.03 and 0.35±0.01 ng/mL when used at concentrations of 50 and 100 μg/mL, respectively, as shown in FIG. 2. Also, the amount of leptin production was significantly lower when the 30% ethanol extract complex of bamboo leaves plus quince was used at a concentration of 100 μg/mL or when the hot-water extract complex of bamboo leaves plus quince was used at a concentration of 50 and 100 μg/mL, compared to when HCA was used at a concentration of 100 μg/mL.

In FIG. 2, Pre represents the pre-adipocytes, C represents the adipocytes, JME represents the 30% ethanol extract of quince and bamboo leaves, and JMW represents the hot-water extract of quince and bamboo leaves.

2-3. Analysis of Glycerol Release

3T3-L1 adipocytes were treated with each of the 30% ethanol extract complex of bamboo leaves plus quince and the hot-water extract complex of bamboo leaves plus quince at concentrations of 50 and 100 μg/mL to measure an amount of glycerol production. An amount of glycerol production in undifferentiated pre-adipocytes was 2.46±1.34 μg/mL, an amount of glycerol production in differentiated adipocytes was 18.10±2.28 μg/mL, and an amount of glycerol production in the positive control was 32.09±1.23 μg/mL when HCA was present at a concentration of 100 μg/m L.

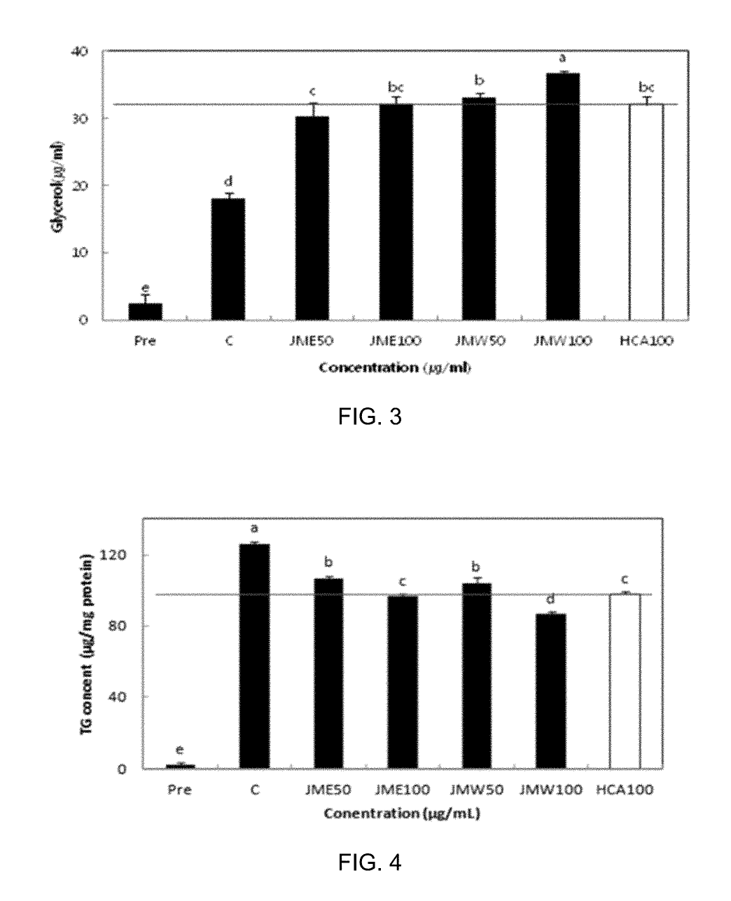

As a result, the amounts of glycerol production in the adipocytes treated with the 30% ethanol extract complex of bamboo leaves plus quince and the hot-water extract complex of bamboo leaves plus quince increased significantly (p<0.05) to 30.34±1.97 and 32.17±1.04 μg/mL, and 33.09±0.63 and 36.61±0.38 μg/mL when used at concentrations of 50 and 100 μg/m L, and the amount of glycerol production was significantly higher when the hot-water extract complex of bamboo leaves plus quince was used at concentrations of 50 and 100 μg/mL, compared to when HCA was used at a concentration of 100 μg/mL, respectively, as shown in FIG. 3.

In FIG. 3, Pre represents the pre-adipocytes, C represents the adipocytes, JME represents the 30% ethanol extract of quince and bamboo leaves, and JMW represents the hot-water extract of quince and bamboo leaves.

These results suggest that the extract complex of bamboo leaves plus quince effectively decomposes triglycerides, which have been stored in the adipocytes, into free fatty acids and glycerol so that the free fatty acids and glycerol are released from the cells.

2-4. Analysis of Triglyceride Content

3T3-L1 adipocytes were treated with each of the 30% ethanol extract complex of bamboo leaves plus quince and the hot-water extract complex of bamboo leaves plus quince at concentrations of 50 and 100 μg/mL to measure a concentration of triglycerides. A content of the triglycerides in undifferentiated pre-adipocytes was 2.14±1.65 μg/mg protein, a content of the triglycerides in differentiated adipocytes was 126.2±1.25 μg/mg protein, and a content of the triglycerides in the positive control was 98.01±1.24 μg/mg protein when HCA was present at a concentration of 100 μg/mL.

As a result, it can be seen that the triglyceride contents in the adipocytes treated with the 30% ethanol extract complex of bamboo leaves plus quince and the hot-water extract complex of bamboo leaves plus quince at the concentrations of 50 and 100 μg/mL decreased significantly (p<0.05) to 106.91±1.44 and 96.75±1.52 μg/mg protein, and 104.39±2.98 and 86.60±1.46 μg/mg protein, respectively, as shown in FIG. 4. Also, the triglyceride content in the adipocytes treated with the 30% ethanol extract complex of bamboo leaves plus quince at a concentration of 100 μg/mL was not significantly different from the triglyceride content in the adipocytes treated with 100 μg/mL of HCA, and the triglyceride content in the adipocytes treated with the hot-water extract complex of bamboo leaves plus quince at a concentration of 100 μg/mL decreased significantly, compared to the triglyceride content in the adipocytes treated with 100 μg/mL of HCA.

In FIG. 4, Pre represents the pre-adipocytes, C represents the adipocytes, JME represents the 30% ethanol extract of quince and bamboo leaves, and JMW represents the hot-water extract of quince and bamboo leaves.

These results suggest that the extract complex of bamboo leaves plus quince effectively reduces the triglyceride content.

### Example 3: In Vivo Animal Experiment

3-1. Analysis of Body Weight Gain, Food Intake, and Food Efficiency Ratio

After laboratory animals were adapted for a week, the hot-water extract complex of bamboo leaves plus quince was orally administered to the C57BL/6J mice for 8 weeks to observe a change in body weight.

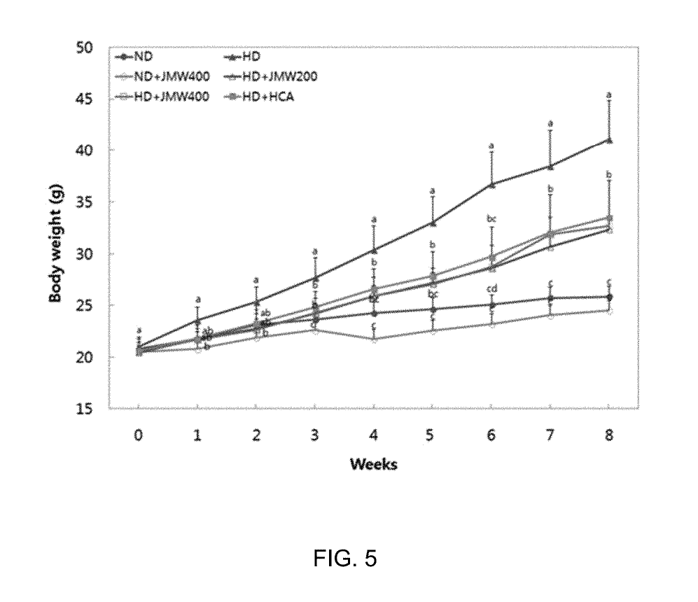

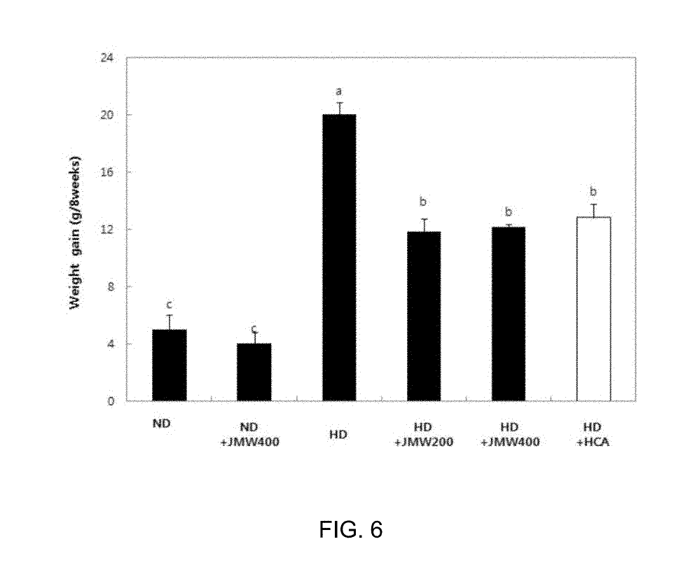

As a result, the changes in body weight were 5.03 g in the case of the ND group, and 4.03 g in the case of ND+JMW400 group (a group in which JMW was orally administered at a dose of 400 mg/kg BW/day), indicating that the change in body weight in the ND+JMW400 group was lower than that in the ND group, but there was no significant difference in the changes in body weight between the two groups, as shown in FIGS. 5 and 6.

On the other hand, the changes in body weight were 20.07 g, 11.88 g, 12.14 g, and 12.83 g in the case of the HD group, the HD+JMW200 group (a group in which JMW was orally administered at a dose of 200 mg/kg BW/day), the HD+JMW400 group (a group in which JMW was orally administered at a dose of 400 mg/kg BW/day), and the HD+HCA group as the positive control, respectively, indicating that there was a significant difference in the changes in body weight between the HD group and the HD+JMW200 and HD+JMW400 groups because the changes in body weight in the HD+JMW200 and HD+JMW400 groups were smaller than that in the HD group and, and the changes in body weight in the HD+JMW200 and HD+JMW400 groups were similar to that in the HD+HCA group.

From these results, it can be seen that the intake of the hot-water extract complex of bamboo leaves plus quince exhibits an effect of inhibiting the body weight gain, and there is no significant difference in the body weight gain according to the dosage of JMW.

Also, as listed in the following Table 2, it was revealed that the food intakes were 4.37 g, 3.89 g, 4.16 g, 3.59 g, 3.80 g, and 3.79 g in the case of the ND group, the ND+JMW400 group, the HD group, the HD+JMW200 group, the HD+JMW400 group, and the HD+HCA group, respectively, indicating that there was a significant difference in the food intake between the ND group and the HD+JMW200 group because the food intake was highest in the ND group and lowest in the HD+JMW200 group. When the food efficiency ratio (FER) was calculated, the food efficiency ratios (FERs) were 1.15, 1.04, 4.82, 3.31, 3.19, and 3.39 in the case of the ND group, the ND+JMW400 group, the HD group, the HD+JMW200 group, the HD+JMW400 group, and the HD+HCA group, respectively, indicating that there was a significant difference in the food intake between the HD+JMW200, HD+JMW400 and HD+HCA groups and the HD group because the food efficiency ratios (FERs) in the HD+JMW200, HD+JMW400 and HD+HCA groups was smaller than that in the HD group.

TABLE 2

| | ND | ND + JMW400 | HD | HD + JMW200 | HD + JMW400 | HD + HCA |
|---|---|---|---|---|---|---|
| Start body | 20.84 ± 0.95^{NS} | 20.49 ± 1.41 | 20.99 ± 1.00 | 20.46 ± 0.83 | 20.55 ± 0.61 | 20.67 ± 0.81 |
| weight (g) | | | | | | |
| Final body | 25.87 ± 1.02^{c} | 24.52 ± 1.49^{^{c}} | 41.06 ± 3.82^{a} | 32.34 ± 2.79^{b} | 32.69 ± 0.98^{b} | 33.50 ± 3.58^{b} |
| weight (g) | | | | | | |
| Weight gain | 5.03 ± 0.98^{c} | 4.03 ± 0.84^{c} | 20.07 ± 0.79^{a} | 11.88 ± 0.89^{b} | 12.14 ± 0.21^{b} | 12.83 ± 0.94^{b} |
| (g/8 weeks) | | | | | | |
| Food intake | 4.37 ± 0.09^{a} | 3.89 ± 0.04^{c} | 4.16 ± 0.08^{b} | 3.59 ± 0.07^{d} | 3.80 ± 0.03^{c} | 3.79 ± 0.05^{c} |
| (g/day) | | | | | | |
| FER | 1.15 ± 0.42^{c} | 1.04 ± 0.60^{c} | 4.82 ± 0.31^{c} | 3.31 ± 0.37^{b} | 3.19 ± 0.13^{b} | 3.39 ± 0.48^{b} |

ND: C57BL/6J mice fed normal diet,
HD: C57BL/6J mice fed high-fat diet,
ND + JMW400: ND and JMW 400 mg/kg BW/day orally administered C57BL/6J mice,
HD + JMW200: HD and JMW 200 mg/kg BW/day orally administered C57BL/6J mice,
HD + JMW400: HD and JMW 400 mg/kg BW/day orally administered C57BL/6J mice,
HD + HCA: HD and HCA 300 mg/kg BW/day orally administered C57BL/6J mice for 8 weeks.
Values are Mean ± SD, n = 10.
^{a-d}Means not sharing a common letter are significantly different among groups (p < 0.05).
NS: Not significant.
FER: Food efficiency ratio = [weight gain (g)]/[total food intake (g) * 100],
JMW: Quinces and Bamboo leaves water extract,
HCA: Hydroxycitric Acid.

3-2. Analysis of Weight of Adipose Tissues

The weights of the adipose tissues in the laboratory animals treated with the hot-water extract complex of bamboo leaves plus quince were measured. The results are listed in the following Table 3.

| | ND | ND + JMW400 | HD | HD + JMW200 | HD + JMW400 | HD + HCA |
|---|---|---|---|---|---|---|
| EAT (g) | 0.53 ± 0.07^{d} | 0.30 ± 0.05^{e} | 2.69 ± 0.04^{a} | 1.40 ± 0.24^{c} | 1.44 ± 0.12^{c} | 1.70 ± 0.14^{b} |
| BAT (g) | 0.08 ± 0.01^{bc} | 0.06 ± 0.01^{c} | 0.18 ± 0.02^{a} | 0.10 ± 0.01^{b} | 0.10 ± 0.01^{b} | 0.09 ± 0.02^{b} |

ND: C57BL/6J mice fed normal diet,
HD: C57BL/6J mice fed high-fat diet,
ND + JMW400: ND and JMW 400 mg/kg BW/day orally administered C57BL/6J mice,
HD + JMW200: HD and JMW 200 mg/kg BW/day orally administered C57BL/6J mice,
HD + JMW400: HD and JMW 400 mg/kg BW/day orally administered C57BL/6J mice,
HD + HCA: HD and HCA 300 mg/kg BW/day orally administered C57BL/6J mice.
Values are Mean ± SD, n = 10.
^{a-e}Means not sharing a common letter are significantly different among groups (p < 0.05).
EAT: Epididymal adipose tissue,
BAT: Brown adipose tissue.
JMW: Quinces and Bamboo leaves water extract,
HCA: Hydroxycitric Acid.

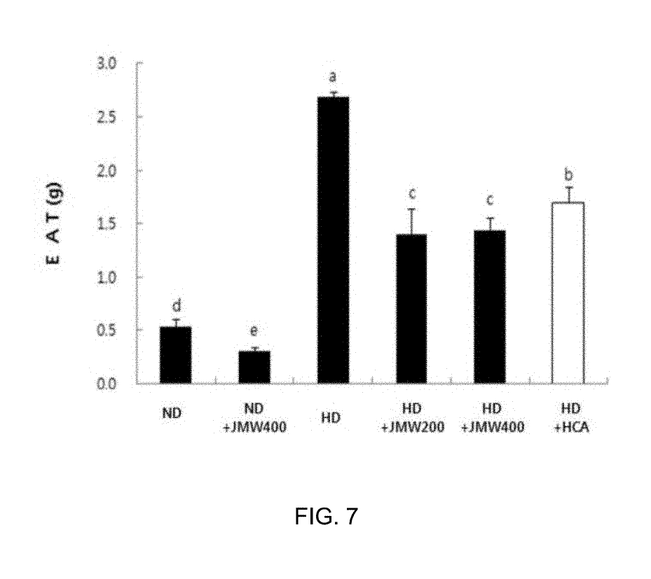

As a result, there was a significant difference in the weight of the epididymal adipose tissue (EAT) between the ND+JMW400 group and the ND group because the weight of the epididymal adipose tissue (EAT) in the ND+JMW400 group was 0.30 g, the value of which was lower than the weight (0.53 g) of the epididymal adipose tissue (EAT) in the ND group. Also, there was a significant difference in the weight of the epididymal adipose tissue (EAT) between the HD+JMW200 and HD+JMW400 groups and the HD group as the obesity control because the weights of the epididymal adipose tissue (EAT) in the HD+JMW200 and HD+JMW400 groups were 1.40 g and 1.44 g, respectively, the values of which were lower than the weight (2.69 g) of the epididymal adipose tissue (EAT) in the HD group. There was no significant difference in the weight of the epididymal adipose tissue (EAT) between the HD+JMW200 group and the HD+JMW400 group, but the weights of the epididymal adipose tissue (EAT) in the two groups were significantly lower than the weight (1.70 g) of the epididymal adipose tissue (EAT) in the HD+HCA group as the positive control (see FIG. 7).

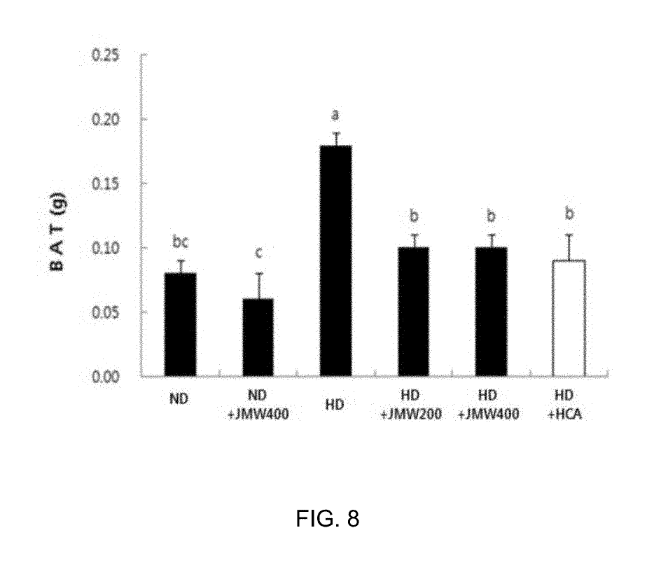

The weight of a brown adipose tissue (BAT) from the back of the neck was analyzed. As a result, there was a significant difference in the weight of the brown adipose tissue (BAT) between the ND+JMW400 group and the ND group because the weight of the brown adipose tissue (BAT) in the ND+JMW400 group was 0.06 g, the value of which was lower than the weight (0.08 g) of the brown adipose tissue (BAT) in the ND group, and there was a significant difference in the weight of the brown adipose tissue (BAT) between the HD+JMW200 and HD+JMW400 groups and the HD group as the obesity control because the weights of the brown adipose tissue (BAT) in the HD+JMW200 and HD+JMW400 groups were 0.10 g and 0.10 g, respectively, the values of which were lower than the weight (0.18 g) of the brown adipose tissue (BAT) in the HD group (see FIG. 8).

From these results, it can be seen that the hot-water extract complex of bamboo leaves plus quince has an effect of reducing the weights of the epididymal adipose tissue and the brown adipose tissue when orally administered.

3-3. Analysis of Blood Lipids

An amount of blood lipids (triglycerides and cholesterol) was measured in the laboratory animals treated with the hot-water extract complex of bamboo leaves plus quince. The results are listed in the following Table 4.

TABLE 4

| | ND | ND + JMW400 | HD | HD + JMW200 | HD + JMW400 | HD + HCA |
|---|---|---|---|---|---|---|
| Triglyceride | 129.33 ± 4.74^{c} | 121.01 ± 6.64^{c} | 200.03 ± 15.15^{a} | 170.43 ± 3.19^{b} | 165.08 ± 10.33^{b} | 174.08 ± 3.49^{b} |
| (mg/dL) | | | | | | |
| Total | 159.08 ± 7.06^{c} | 149.77 ± 10.55^{c} | 260.55 ± 1.04^{a} | 240.88 ± 8.48^{b} | 237.25 ± 7.89^{b} | 241.53 ± 8.87^{b} |
| cholesterol | | | | | | |
| (mg/dL) | | | | | | |
| HDL- | 62.06 ± 8.04^{c} | 83.63 ± 3.45^{b} | 37.70 ± 1.41^{a} | 78.48 ± 2.36^{b} | 79.41 ± 5.26^{b} | 75.66 ± 8.11^{b} |
| cholesterol | | | | | | |
| (mg/dL) | | | | | | |
| LDL- | 71.15 ± 6.61^{c} | 41.94 ± 8.54^{d} | 182.84 ± 5.86^{a} | 128.32 ± 6.29^{b} | 124.82 ± 7.44^{b} | 131.06 ± 6.82^{b} |
| cholesterol | | | | | | |
| (mg/dL) | | | | | | |

ND: C57BL/6J mice fed normal diet,
HD: C57BL/6J mice fed high-fat diet,
ND + JMW400: ND and JMW 400 mg/kg BW/day orally administered C57BL/6J mice,
HD + JMW200: HD and JMW 200 mg/kg BW/day orally administered C57BL/6J mice,
HD + JMW400: HD and JMW 400 mg/kg BW/day orally administered C57BL/6J mice.
HD + HCA: HD and HCA 300 mg/kg BW/day orally administered C57BL/6J mice.
Values are Mean ± SD, n = 10.
^{a-d}Means not sharing a common letter are significantly different among groups (p < 0.05).
JMW: Quinces and Bamboo leaves water extract,
HCA: Hydroxycitric Acid.

The concentrations of the blood triglycerides were 129.33 mg/dL and 121.01 mg/dL in the case of the ND group and the ND+JMW400 group, respectively. The concentration of the blood triglycerides was shown to be high at 200.03 mg/dL in the case of the HD group, which was significantly reduced to 170.43 mg/dL and 165.08 mg/dL in the HD+JMW200 group and the HD+JMW400 group, respectively. There was no significant difference in the concentration of the blood triglycerides between the HD+HCA group and the HD+JMW200 and HD+JMW400 groups because the concentration of the blood triglycerides was 174.08 mg/dL in the HD+HCA group.

There was no significant difference in total blood cholesterol between the ND group and the ND+JMW400 group because concentrations of the total blood cholesterol were 159.08 mg/dL and 149.77 mg/dL in the case of the ND group and the ND+JMW400 group, respectively. On the other hand, the concentration of the total blood cholesterol was shown to be high at 260.55 mg/dL in the case of the HD group, which was significantly reduced to 240.88 mg/dL and 237.25 mg/dL in the case of the HD+JMW200 group and the HD+JMW400 group, respectively. Also, there was no significant difference in concentration of the total blood cholesterol between the HD+HCA group and the HD+JMW200 and HD+JMW400 groups because the concentration of the total blood cholesterol was 241.53 mg/dL in the case of the HD+HCA group.

An LDL-cholesterol concentration in the ND group was 71.15 mg/dL, and an LDL-cholesterol concentration in the ND+JMW400 group was 41.94 mg/dL, the value of which was significantly lower than that in the ND group. The LDL-cholesterol concentration was shown to be highest at 182.84 mg/dL in the case of the HD group as the obesity control, and the LDL-cholesterol concentrations were shown to be 128.32 mg/dL and 124.82 mg/dL in the case of the HD+JMW200 group and the HD+JMW400 group, respectively, the values of which were shown to be significantly lower than that in the HD group. There was no significant difference in the LDL-cholesterol concentration between the positive control and the HD+JMW200 and HD+JMW400 groups because the LDL-cholesterol concentration was 131.06 mg/dL in the case of the positive control.

An HDL-cholesterol concentration in the ND group was 62.06 mg/dL, and an HDL-cholesterol concentration in the ND+JMW400 group was 83.63 mg/dL the value of which was significantly higher than that in the ND group. The HDL-cholesterol concentration was shown to be lowest at 37.70 mg/dL in the case of the HD group, and the HDL-cholesterol concentrations were shown to be 78.48 mg/dL and 79.41 mg/dL in the case of the HD+JMW200 and 400 groups, respectively, the values of which were significantly higher than that in the HD group. Also, there was no significant difference in the HDL-cholesterol concentration between the HD+HCA group and the JMW200 and 400 groups because the HDL-cholesterol concentration was 75.66 mg/dL in the case of the HD+HCA group.

Therefore, it can be seen that the total cholesterol concentration, the blood triglyceride concentration, and the LDL-cholesterol concentration are reduced when JMW is orally administered, and blood lipid metabolism in the C57BL/6J mice as an animal obesity model is improved with an increase in the HDL-cholesterol concentration.

3-4. Analysis of Adipose Tissues and Adipocytes

The analysis of adipose tissues was performed by the Research Institute of Medical Science of Korea. The adipocytes were observed at a magnification of 400×, and all fats were observed at 20 designated sites at a magnification of 400× for the purpose of identity. The size of cells was expressed as a diameter, and the area of the cells was expressed as nm². In this case, the area of the cells was calculated using Image Pro Plus.

Specifically, a method of measurement was performed by first fixing an extracted adipose tissue in a formalin solution for 24 hours or more and washing the adipose tissue with running water to remove the formalin. A paraffin block, which was prepared by impregnating paraffin into the sufficiently washed adipose tissue using a Thermo tissue processor and embedding the paraffin into the adipose tissue at the Thermo embedding center, was cut into 4-μm sections using a microtome, attached onto a slide, and then dried. After the paraffin was removed from the adipose tissue-attached slide using xylene, the adipose tissue was stained with hematoxylin and eosin (H&E), and a histological change of the adipose tissue was observed at a magnification of 400× under an optical microscope.

(1) Size and Area of Visceral Adipose Tissue

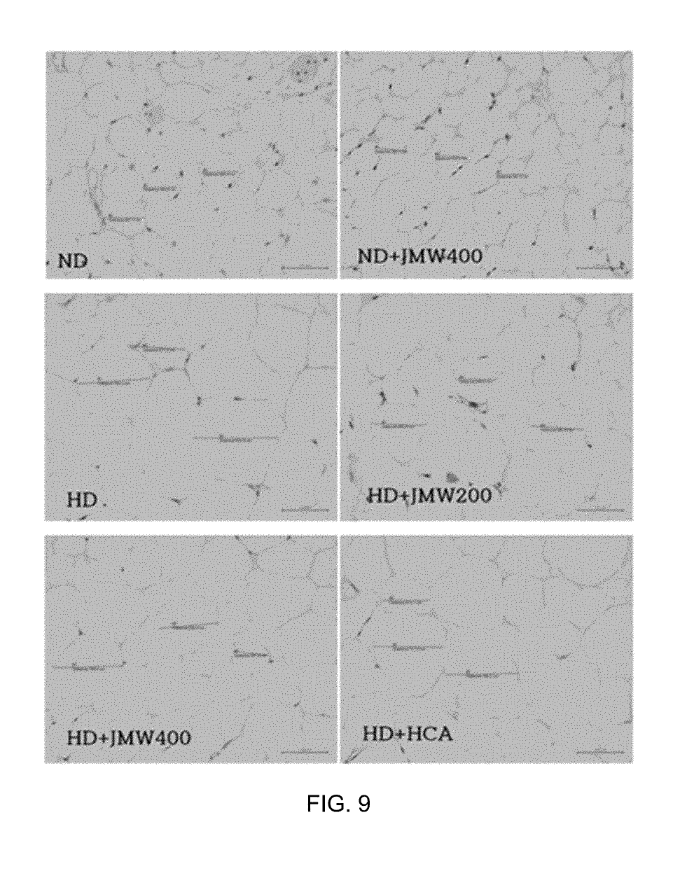

To observe the size of lipid droplets, an animal was dissected, and a visceral adipose tissue was then stained with hematoxylin & eosin (H&E). As a result, it can be seen that the size of the visceral adipose tissue of the laboratory animal increased when observed with the naked eye in the group in which a 60% high fat diet was supplied for 8 weeks, compared to the group in which the normal diet was supplied, indicating that obesity was induced, as shown in FIG. 9.

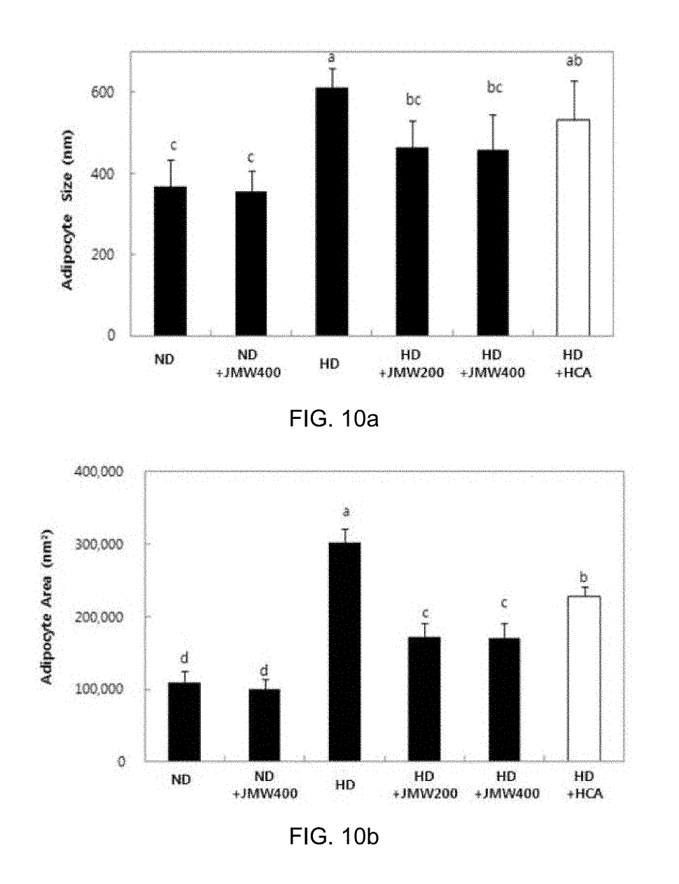

Also, there was no significant difference in the size of the visceral adipose tissue between the ND group and the ND+JMW400 group because the sizes of the visceral adipose tissue were 368.0 nm and 354.5 nm in the case of the ND group and the ND+JMW400 group, respectively, as shown in FIG. 10a. The size of the visceral adipose tissue was largest (610.3 nm) in the HD group as the obesity control, and the sizes of the visceral adipose tissues in the HD+JMW200 and 400 groups were 463.7 nm and 458.9 nm, respectively, the values of which were significantly lower than that in the obesity control. In the HD+HCA group as the positive control, the size of the visceral adipose tissue was 530.4 nm, the value of which was significantly higher than those of the HD+JMW200 and 400 groups.

Further, there was no significant difference in the area of the visceral adipose tissue between the ND group and the ND+JMW400 group because the areas of the visceral adipose tissue were 109,724.3 nm² and 100,836.0 nm² in the case of the ND group and the ND+JMW400 group, respectively, as shown in FIG. 10b. The area of the visceral adipose tissue was widest at 302,347.1 nm² in the case of the HD group as the obesity control, and the areas of the visceral adipose tissues in the HD+JMW200 and 400 groups were 172,173.9 nm² and 171,082.8 nm², respectively, the values of which were significantly lower than that of the obesity control. There was no significant difference in the area of the visceral adipose tissue between the respective sample groups, and the area of the visceral adipose tissue in the HD+HCA group as the positive control was 228,3943.9 nm², the value of which was significantly wider than those of the HD+JMW200 and 400 groups.

Therefore, it can be seen that the extract complex of bamboo leaves plus quince has an effect of reducing the size and area of the visceral adipose tissue in the groups in which the extract complex of bamboo leaves plus quince is ingested.

(2) Size and Area of Brown Adipose Tissue

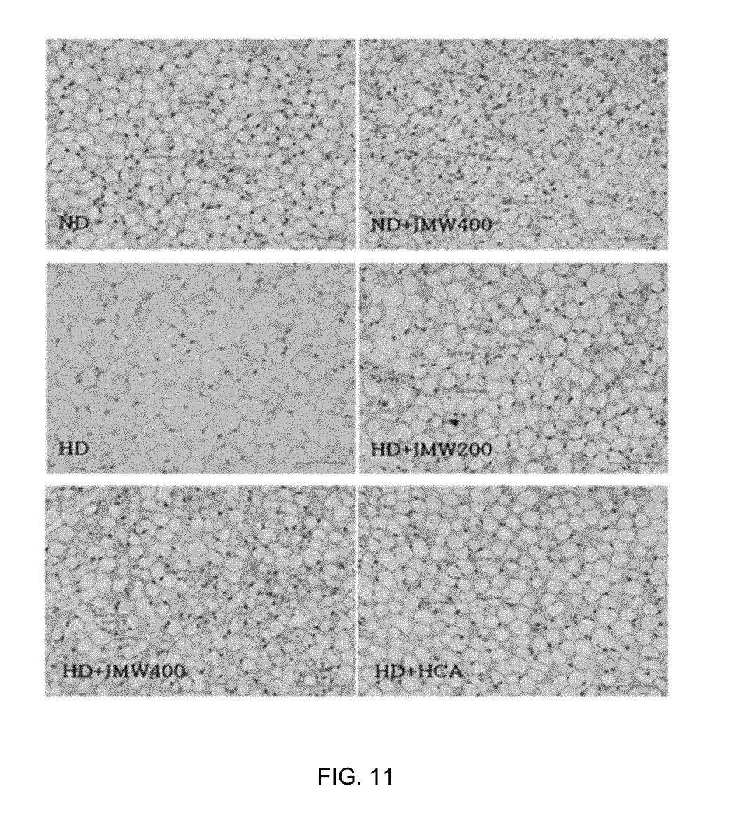

To observe the size of lipid droplets, an animal was dissected, and a brown adipose tissue was then stained with hematoxylin & eosin (H&E). As a result, it can be seen that the size of the brown adipose tissue of the laboratory animal increased when observed with the naked eye in the group in which the 60% high fat diet was supplied for 8 weeks, compared to the group in which the normal diet was supplied, indicating that obesity was induced, as shown in FIG. 11.

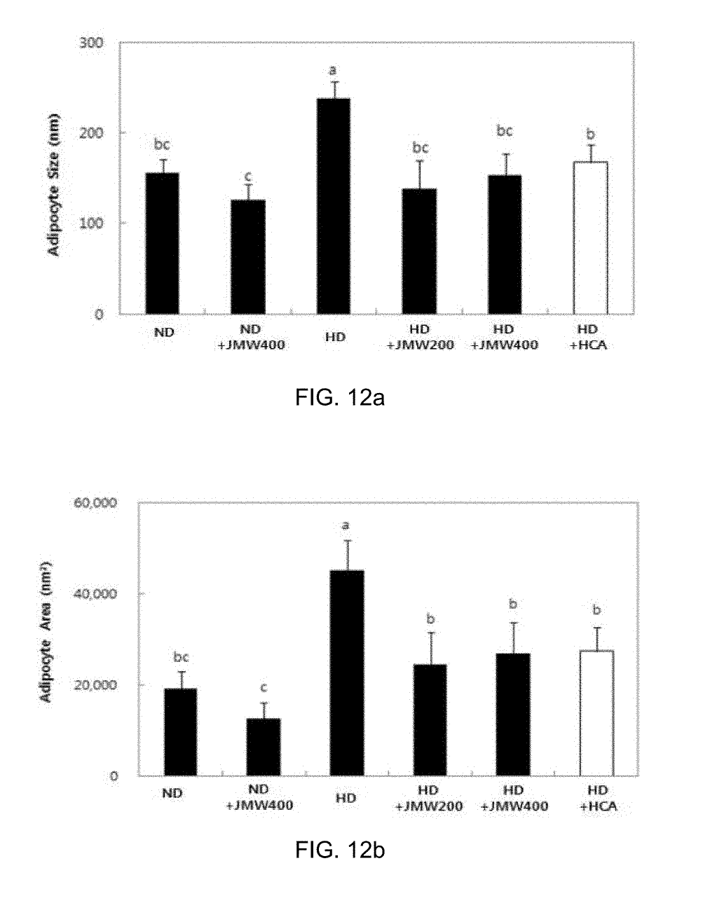

Also, the size of the brown adipose tissue in the ND group was 155.8 nm, and the size of the brown adipose tissue in the ND+JMW400 group was 125.5 nm, the value of which was significantly lower than that in the ND group, as shown in FIG. 12a. The size of the brown adipose tissue was largest (238.2 nm) in the HD group as the obesity control, and the sizes of the brown adipose tissues in the HD+JMW200 and 400 groups were 138.0 nm and 153.0 nm, respectively, the values of which were significantly lower than that in the obesity control. There was no significant difference in the size of the brown adipose tissue between the sample-added groups, and the size of the brown adipose tissue in the HD+HCA group as the positive control was 167.7 nm, the value of which was significantly higher than those in the HD+JMW200 and 400 groups.

Further, the area of the brown adipose tissue in the ND group was 19,218.4 nm², and the area of the brown adipose tissue in the ND+JMW400 group was 12,594.4 nm², the value of which was significantly lower than that in the ND group, as shown in FIG. 12b. The area of the brown adipose tissue was widest (45,019.5 nm²) in the HD group as the obesity control, and the areas of the brown adipose tissues in the HD+JMW200 and 400 groups were 24,362.4 nm² and 26,814.9 nm², respectively, the values of which were significantly lower than that in the obesity control. The area of the brown adipose tissue in the HD+HCA group as the positive control was 27,355.8 nm², the value of which was not significantly different from those in the HD+JMW200 and 400 groups.

Therefore, it can be seen that the extract complex of bamboo leaves plus quince has an effect of reducing the size and area of the brown adipose tissue in the groups in which the extract complex of bamboo leaves plus quince is ingested.

(3) Size and Area of Epididymal Adipose Tissue

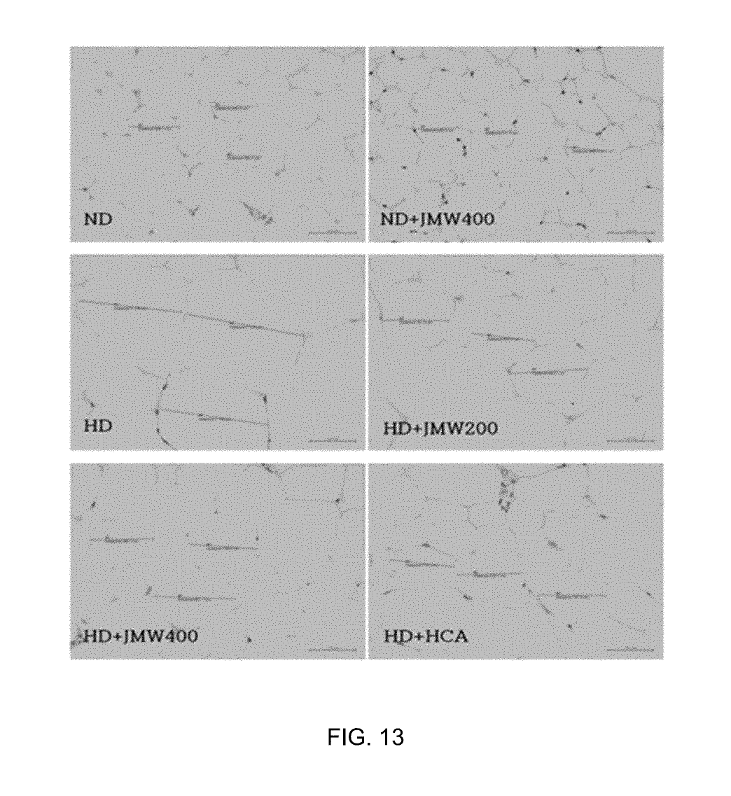

To observe the size of lipid droplets, an animal was dissected, and an epididymal adipose tissue was then stained with hematoxylin & eosin (H&E). As a result, it can be seen that the size of the epididymal adipose tissue of the laboratory animal increased when observed with the naked eye in the group in which the 60% high fat diet was supplied for 8 weeks, compared to the group in which the normal diet was supplied, indicating that obesity was induced, as shown in FIG. 13.

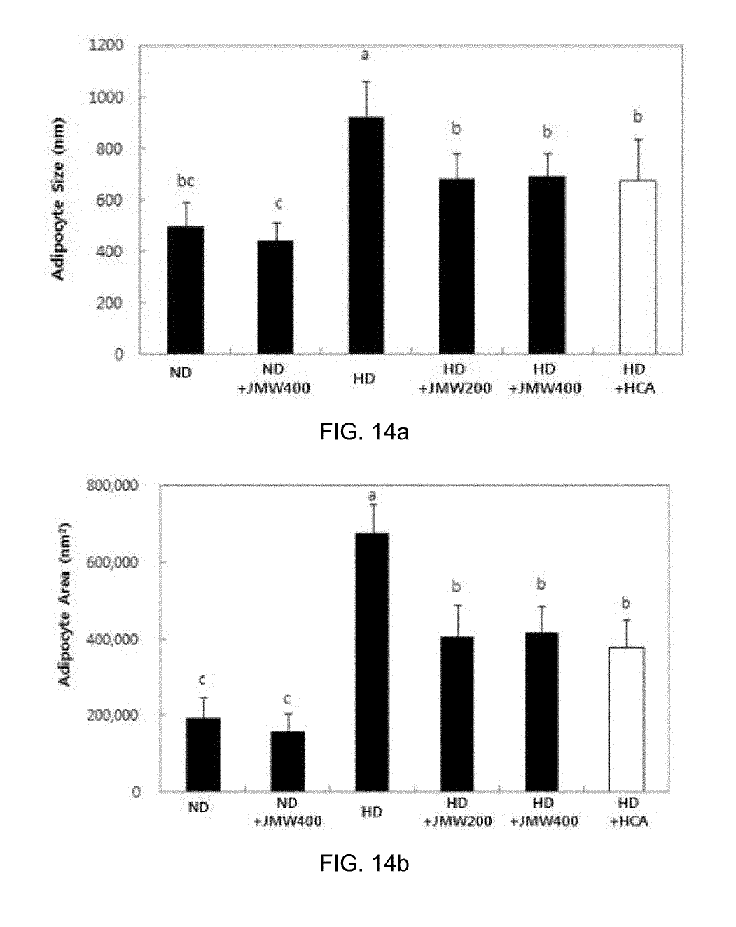

Also, the size of the epididymal adipose tissue in the ND group was 498.2 nm, and the size of the epididymal adipose tissue in the ND+JMW400 group was 443.2 nm, the value of which was significantly lower than that in the ND group, as shown in FIG. 14a. The size of the epididymal adipose tissue was largest (919.3 nm) in the HD group as the obesity control, and the sizes of the epididymal adipose tissues in the HD+JMW200 and 400 groups were 679.9 nm and 689.5 nm, respectively, the values of which were significantly lower than that in the obesity control. The size of the epididymal adipose tissue in the HD+HCA group as the positive control was 675.6 nm, the value of which was not significantly different from those in the HD+JMW200 and 400 groups.

Further, the area of the epididymal adipose tissue in the ND group was 193,683.3 nm², and the area of the epididymal adipose tissue in the ND+JMW400 group was 157,907.5 nm², the value of which was not significantly different from that in the ND group, as shown in FIG. 14b. The area of the epididymal adipose tissue was widest (678,649.1 nm²) in the HD group as the obesity control, and the areas of the epididymal adipose tissues in the HD+JMW200 and 400 groups were 408,477.2 nm² and 416,554.2 nm², respectively, the values of which were significantly lower than that in the obesity control. The area of the brown adipose tissue in the HD+HCA group as the positive control was 377,630.7 nm², the value of which was not significantly different from those in the HD+JMW200 and 400 groups.

Therefore, it can be seen that the extract complex of bamboo leaves plus quince has an effect of reducing the size and area of the epididymal adipose tissue in the groups in which the extract complex of bamboo leaves plus quince is ingested.

Because the extract complex of bamboo leaves and quince according to the present disclosure has an excellent anti-obesity effect without causing any side effects, the extract complex can be effectively used in a pharmaceutical composition and health food for preventing, improving and treating obesity. Therefore, a novel therapeutic agent whose effect goes beyond conventional natural anti-obesity drugs and a novel therapy can be provided according to the present disclosure.

Also, when quince and bamboo leaves are combined, a bitter taste of the bamboo leaves can be reduced due to the quince, and the extract complex of bamboo leaves and quince can exhibit an effect of improving obesity even when used at a concentration lower than the sum of the effective concentrations of the quince and bamboo leaf single extracts. Therefore, when the extract complex is used at an effective concentration, the problems such as a difficulty in formulation and preparation can be solved due to the low concentration of sugar, compared to the single extracts.

Further, an equivalent amount of each of the extracts should be generally used so that the extract complex exhibits the same effect as the single extract. However, the extract complex according to the present disclosure can exhibit the same effect even when half of the effective dose of the complex extract is ingested, compared to when the single extracts are ingested.

It will be apparent to those skilled in the art that various modifications can be made to the above-described exemplary embodiments of the present disclosure without departing from the scope of the invention. Thus, it is intended that the present disclosure covers all such modifications provided they come within the scope of the appended claims and their equivalents.

What is claimed is:

1. A method of treating obesity, comprising:
administering an extract complex of bamboo leaves and quince to a subject.

2. The method of claim 1, wherein the extraction is performed using water, a C1 to C4 lower alcohol, or a mixed solvent thereof.

3. The method of claim 2, wherein the lower alcohol is ethanol.

4. The method of claim 1, wherein the extract complex is obtained by mixing a bamboo leaf extract and a quince extract at a weight ratio of 1:0.1 to 10.

5. The method of claim 4, wherein the weight ratio is 1:1.

6. A method of improving obesity, comprising:
administering an extract complex of bamboo leaves and quince to a subject.

7. The method of claim 6, wherein the extraction is performed using water, a C1 to C4 lower alcohol, or a mixed solvent thereof.

8. The method of claim 7, wherein the lower alcohol is ethanol.

9. The method of claim 6, wherein the extract complex is obtained by mixing a bamboo leaf extract and a quince extract at a weight ratio of 1:0.1 to 10.

10. The method of claim 9, wherein the weight ratio is 1:1.
